(19) United States
(12) Patent Application Publication
(10) Pub. No.: US 2026/0347846 A1
(43) Pub. Date: Nov. 5, 2026

(54) METHODS AND COMPOSITIONS FOR HYPERCHOLESTEROLEMIA AND RELATED DISORDERS

(71) Applicant: Galatea Bio, Inc., Miami Lakes, FL (US)

(72) Inventor: Nicholas KATSANIS, Durham, NC (US)

(21) Appl. No.: 19/334,100

(22) Filed: Sep. 19, 2025

Related U.S. Application Data
(60) Provisional application No. 63/697,368, filed on Sep. 20, 2024.

(51) Int. Cl. C12N 15/113; A61K 31/712; A61K 31/7125; A61P 3/06

(52) U.S. Cl. CPC C12N 15/113; A61K 31/712; A61K 31/7125; A61P 3/06; C12N 2310/14; C12N 2310/3125; C12N 2310/315; C12N 2310/321; C12N 2310/322; C12N 2310/3231; C12N 2320/30

(57) ABSTRACT

Described are methods directed towards analysis of disease and administering a nucleic acid molecule or a salt thereof configured to hybridize to a PDZK1 sequence to subject.

### CROSS-REFERENCE

This application claims the benefit of priority to U.S. Provisional Application No. 63/697,368 filed Sep. 20, 2024, which is incorporated by reference herein in its entirety.

### SEQUENCE LISTING

The material in the XML file, titled ‘60115-707.201.xml’, created on Jul. 8, 2026, and having a size of 28,577 bytes, is incorporated by reference herein.

### BACKGROUND

Familial hypercholesterolemia (FH) is an inherited lipid disorder that can be characterized by extremely high levels of low-density lipoprotein cholesterol in the blood with concomitantly low levels of high-density cholesterol. The disorder has an observed population frequency of 1:300 and is often transmitted as a dominant trait, although recessive FH has also been reported with an earlier onset and severely more aggressive symptomology and progression. Undiagnosed and untreated FH can lead to life-threatening complications, most prominently for coronary heart disease (50% chance before the age of 50 for men and 40% risk for women by the age of 60.

### SUMMARY

In an aspect, the present disclosure provides a method of treating a subject having or suspected of having hypertension or hyperlipidemia, comprising administering a nucleic acid molecule or a salt thereof configured to hybridize to a PDZK1 sequence to the subject.

In some embodiments, the nucleic acid molecule or salt thereof comprises a short interfering RNA (siRNA) molecule, a short hairpin RNA (shRNA) molecule, a microRNA adapted shRNA (shRNAmiR) molecule, a microRNA (miRNA molecule), or an antisense oligonucleotide (ASO), or any combination thereof.

In some embodiments, the nucleic acid molecule or salt thereof comprises a siRNA molecule, wherein the siRNA molecule comprises a duplex region of about 18 to about 27 base pairs.

In some embodiments, the PDZK1 sequence is a PDZKI mRNA sequence.

In some embodiments, the nucleic acid molecule is not encapsulated in an exosome.

In some embodiments, the nucleic acid molecule comprises a sequence having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%, or 100% complementarity or identity to at least 18-27 consecutive nucleotides of SEQ ID NO: 29.

In some embodiments, the nucleic acid molecule comprises a sequence having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%, or 100% sequence identity to any one of SEQ ID NOs: 1-28.

In some embodiments, the subject has or is suspected of having hyperlipidemia, wherein the hyperlipidemia comprises hypercholesterolemia.

In some embodiments, the hypercholesterolemia comprises a serum LDL level of greater than about 125 mg/dL.

In some embodiments, the hypercholesterolemia comprises a serum LDL: serum HDL ratio of less than about 3.5:1.

In some embodiments, the subject has or is suspected of having hyperlipidemia, wherein the hyperlipidemia comprises glyceridemia.

In some embodiments, the glyceridemia comprises hypertriglyceridemia.

In some embodiments, the hypertriglyceridemia comprises a serum triglyceride level of greater than about 150 mg/dL.

In some embodiments, the subject has or is suspected of having hypertension.

A method of treating a subject having or suspected of having glyceridemia, comprising administering a nucleic acid molecule or a salt thereof to the subject, wherein the nucleic acid molecule comprises a sequence having at least 80% sequence identity to any one of SEQ ID NOs: 1-28, or a variant thereof.

In some embodiments, the glyceridemia comprises hypertriglyceridemia.

A method of treating a subject having or suspected of having hyperlipidemia, comprising administering a nucleic acid molecule or a salt thereof to the subject, wherein the nucleic acid molecule comprises a sequence having at least 80% sequence identity to any one of SEQ ID NOs: 1, 2, 7, or 10-12, 14-28, or a variant thereof.

In some embodiments, the hyperlipidemia comprises hypercholesterolemia.

In some embodiments, the hyperlipidemia comprises hypertriglyceridemia.

A method of treating a subject having or suspected of having hypertension, comprising administering a nucleic acid molecule or a salt thereof to the subject, wherein the nucleic acid molecule comprises a sequence having at least 80% sequence identity to any one of SEQ ID NOs: 1, 2, 7, or 10-28, or a variant thereof.

In some embodiments, the nucleic acid molecule is an siRNA molecule comprising a duplex region.

In some embodiments, the duplex region is 18-27 base pairs in length.

An siRNA molecule or a salt thereof having a duplex region 18-27 base pairs in length, wherein the duplex region comprises a sequence having at least 80% sequence identity to any one of SEQ ID NOs: 2, 15-22, 24-26, or 28.

An siRNA molecule, or a salt thereof having a duplex region 18-27 base pairs in length, wherein the duplex region comprises a sequence having at least 90% sequence identity to any one of SEQ ID NOs: 2, 7, 11, 15-22, or 23-28, or a variant thereof.

An siRNA molecule or a salt thereof having a duplex region 18-27 base pairs in length, wherein the duplex region comprises a sequence having at least 91% sequence identity to any one of SEQ ID NOs: 2, 3, 4, 7, 8, 9, 11, 15-22, or 23-28, or a variant thereof.

An siRNA molecule or a salt thereof having a duplex region 18-27 base pairs in length, wherein the duplex region comprises a sequence having at least 96% sequence identity to any one of SEQ ID NOs: 2, 3, 4, 7, 8, 9, 11, 12, 15-22, or 23-28, or a variant thereof.

In some embodiments, the duplex region is 20-27 base pairs in length.

An siRNA molecule or a salt thereof having a duplex region 20-27 base pairs in length, wherein the duplex region comprises a sequence having at least 80% sequence identity to any one of SEQ ID NOs: 1, 2, 3, 4, 8, 9, or 15-22, or 23-28, or a variant thereof.

In some embodiments, the siRNA molecule comprises one or more non-natural RNA nucleotide or one or more non-natural linkage.

In some embodiments, the siRNA molecule comprises one or more non-natural RNA nucleotide, wherein the one or more non-natural RNA nucleotide comprises 2′-fluoro, 2′-O-methyl, 21-O-methyl, 21-O-methoxy-ethyl, 21-O-methoxy-ethoxy, 5′-methyl, SNA, hGNA, hhGNA, mGNA, TNA, h′GNA, a locked nucleic acid (LNA), or any combination thereof.

In some embodiments, the siRNA molecule comprises one or more non-natural linkage, wherein the one or more non-natural linkage comprises a phosphorothioate or methylphosphonate linkage.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings (also “Figure” and “FIG.” herein), of which:

FIG. 1 illustrates an example of a schematic representation of duplication.

FIG. 2A shows assay data results for variant rs2976384 in Assay 1 through Assay 5.

FIG. 2B shows assay data results for variant rs2976384 in Assay 6 through Assay 10.

FIG. 3A shows a phenome-wide view for variant rs2976384 showing phenome-wide significant association with traits from the UK Biobank including hypertension.

FIG. 3B shows a phenome-wide view for variant rs2976384 showing phenome-wide significant association with traits from the UK Biobank including essential hypertension.

FIG. 3C shows a phenome-wide view for variant rs2976384 showing phenome-wide significant association with traits from the UK Biobank including hyperglyceridemia.

FIG. 4 illustrates an example of a computing device; in this case, a device with one or more processors, memory, storage, and a network interface.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

### TERMS AND DEFINITIONS

As used herein, the singular forms “a,” “an,” and “the” include plural references unless the context clearly dictates otherwise. Any reference to “or” herein is intended to encompass “and/or” unless otherwise stated.

As used herein, the phrases “at least one,” “one or more,” and “and/or” are open-ended expressions that are both conjunctive and disjunctive in operation. For example, each of the expressions “at least one of A, B and C,” “at least one of A, B, or C,” “one or more of A, B, and C”, “one or more of A, B, or C” and “A, B, and/or C” means A alone, B alone, C alone, A and B together, A and C together, B and C together, or A, B and C together. As used herein, the phrase “at most three” can mean less than one, one, two, or three.

Reference throughout this specification to “some embodiments,” “further embodiments,” or “a particular embodiment,” means that a particular feature, structure, or characteristic described in connection with the embodiment is included in at least one embodiment. Thus, the appearances of the phrase “in some embodiments,” or “in further embodiments,” or “in a particular embodiment” in various places throughout this specification are not necessarily all referring to the same embodiment. Furthermore, the particular features, structures, or characteristics may be combined in any suitable manner in one or more embodiments

The terms “subject,” “individual,” and “patient” may be used interchangeably and refer to humans, as well as non-human mammals (e.g., non-human primates, canines, equines, felines, porcines, bovines, ungulates, lagomorphs, rodents, and the like). In various embodiments, the subject can be a human (e.g., adult male, adult female, adolescent male, adolescent female, male child, female child) under the care of a physician or other health worker in a hospital, as an outpatient, or other clinical context. In certain embodiments, the subject may not be under the care or prescription of a physician or other health worker. In some embodiments, the subject may be under the care of a dental professional.

As used herein, “treatment” or “treating” refers to an approach for obtaining beneficial or desired results with respect to a disease, disorder, or medical condition including, but not limited to, a therapeutic benefit and/or a prophylactic benefit. In certain embodiments, treatment or treating involves administering a therapeutic to a subject. A therapeutic benefit may include the eradication or amelioration of the underlying disorder being treated. Also, a therapeutic benefit may be achieved with the eradication or amelioration of one or more of the physiological symptoms associated with the underlying disorder, such as observing an improvement in the subject, notwithstanding that the subject may still be afflicted with the underlying disorder.

Certain ranges are presented herein with numerical values being preceded by the term “about.” The term “about” is generally used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Reference to an item in the singular is generally to be understood as including the plural and vice versa unless explicitly stated otherwise or clear from the text. Grammatical conjunctions are generally intended to express any and all disjunctive conjunctions and conjunctions of conjunctive clauses, sentences, words, and the like, unless otherwise stated or clear from the context. Thus, the term “or” is generally understood to be an inclusive “or” encompassing both alternatives.

The term “nucleic acid,” generally refers to a polymeric form of nucleotides of any length, either ribonucleotides or deoxynucleotides. Thus, this term includes, but is not limited to, single-, double-, or multi-stranded DNA or RNA, genomic DNA, cDNA, DNA-RNA hybrids, or a polymer including purine and pyrimidine bases or other natural, chemically or biochemically modified, non-natural, or derivatized nucleotide bases. The term “nucleic acid” generally is understood to include, as applicable to the embodiment being described, single-stranded (such as sense or antisense) and double-stranded polynucleotides.

Percent identity or complementarity between particular stretches of nucleic acid sequences or within nucleic acids described herein can be determined using any convenient method. Example methods include BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), e.g., using default settings, which uses the algorithm of Smith and Waterman (Adv. Appl. Math., 1981, 2, 482-489).

As used herein, the term “siRNA” generally refers to a double stranded nucleic acid in which the strands comprise RNA, RNA analog(s) or RNA and DNA. The siRNA can comprise from about 18 to about 40 nucleotides. The siRNA can have overhangs (e.g. 2 bp overhangs) on the 4′ ends of the strands such that the duplex region in the siRNA comprises 17-27 nucleotides. In some embodiments, the antisense strand of the siRNA is sufficiently complementary with a target or predetermined sequence to mediate gene silencing of the target or predetermined sequence within a cell.

siRNA can generally be provided in several forms including, e.g., as one or more isolated small-interfering RNA (siRNA) duplexes, as longer double-stranded RNA (dsRNA), or as siRNA or dsRNA transcribed from a transcriptional cassette in a DNA plasmid. The siRNA sequences may have overhangs (e.g., 4′ or 5′ overhangs as described in Elbashir et al., Genes Dev., 15:188 (2001) or Nykanen et al., Cell, 107:309 (2001), or may lack overhangs (i.e., to have blunt ends).

As used herein, the term “shRNA” (small hairpin RNA) generally refers to an RNA molecule comprising an antisense region, a loop portion and a sense region, wherein the sense region has complementary nucleotides that can base pair with the antisense region to form a duplex stem. Following post-transcriptional processing, the small hairpin RNA can be converted into a small interfering RNA (siRNA) by a cleavage event mediated by the enzyme Dicer, which is a member of the RNase III family.

In some embodiments, a nucleic acid as described herein comprises one or more non-natural linkages or one or more non-natural nucleotides. Non-natural nucleotides can include, but are not limited to, 2′-fluoro, 2′-O-methyl, 2′-O-methyl, 2′-O-methoxy-ethyl, 2′-O-methoxy-ethoxy, 5′-methyl, SNA, hGNA, hhGNA, mGNA, TNA, h′GNA, locked nucleic acids (LNAs), GNA-isoC, GNA-isoG, 5′-mUNA, 4′-mUNA, 4′-mUNA, 2′-mUNA, or an abasic nucleotide (e.g. DNA or RNA). Non-natural linkages can include, but are not limited to, phosphorothioate and methylphosphonate. In some embodiments, an oligonucleotide as described herein comprises a modified uracil. Example nucleobases and nucleosides having a modified uracil include pseudouridine (T), pyridin-4-one ribonucleoside, 5-aza-uridine, 6-aza-uridine, 2-thio-5-aza-uridine, 2-thio-uridine (s2U), 4-thio-uridine (s4U), 4-thio-pseudouridine, 2-thio-pseudouridine, 5-hydroxy-uridine (ho5U), 5-aminoallyl-uridine, 5-halo-uridine (e.g., 5-iodo-uridine or 5-bromo-uridine), 4-methyl-uridine (m3U), 5-methoxy-uridine (mo5U), uridine 5-oxyacetic acid (cmo5U), uridine 5-oxyacetic acid methyl ester (mcmo5U), 5-carboxymethyl-uridine (cm5U), 1-carboxymethyl-pseudouridine, 5-carboxyhydroxymethyl-uridine (chm5U), 5-carboxyhydroxymethyl-uridine methyl ester (mchm5U), 5-methoxycarbonylmethyl-uridine (mcm5U), 5-methoxycarbonylmethyl-2-thio-uridine (mcm5s2U), 5-aminomethyl-2-thio-uridine (nm5s2U), 5-methylaminomethyl-uridine (mnm5U), 5-methylaminomethyl-2-thio-uridine (mnm5s2U), 5-methyl aminomethyl-2-seleno-uridine (mnm5se2U), 5-carbamoylmethyl-uridine (ncm5U), 5-carboxymethylaminomethyl-uridine (cmnm5U), 5-carboxymethylaminomethyl-2-thio-uridine (cmnm5s2U), 5-propynyl-uridine, 1-propynyl-pseudouridine, 5-taurinomethyl-uridine (Tm5U), 1-taurinomethyl-pseudouridine, 5-taurinomethy 1-2-thio-uridine (Tm5s2U), 1-taurinomethyl-4-thio-pseudouridine, 5-methyl-uridine (m5U, i.e., having the nucleobase deoxythymine), 1-methylpseudouridine (m1w), 5-methyl-2-thio-uridine (m5s2U), 1-methyl-4-thio-pseudouridine (m1s4w), 4-thio-1-methyl-pseudouridine, 4-methyl-pseudouridine (m3w), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m5D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine (aka 1-methylpseudouridine (m1w)), 4-(3-amino-3-carboxypropyl) uridine (acp3U), 1-methyl-3-(3-amino-3-carboxypropyl) pseudouridine (acp3 w), 5-(isopentenylaminomethyl) uridine (inm5U), 5-(isopentenylaminomethyl)-2-thio-uridine (inm5s2U), a-thio-uridine, 2′-O-methyl-uridine (Um), 5,2′-O-dimethyl-uridine (m5Um), 2′-O-methyl-pseudouridine (wm), 2-thio-21-O-methyl-uridine (s2Um), 5-methoxycarbonylmethy 1-21-O-methyl-uridine (mcm5Um), 5-carbamoylmethy 1-21-O-methyl-uridine (ncm5Um), 5-carboxymethylaminomethy 1-21-O-methyl-uridine (cmnm5Um), 4,2′-O-dimethyl-uridine (m3Um), 5-(isopentenylaminomethyl)-21-O-methyl-uridine (inm5Um), 1-thio-uridine, deoxythymidine, 2′-F-ara-uridine, 2′-F-uridine, 2′-OH-ara-uridine, 5-(2-carbomethoxyvinyl) uridine, and 5-[3-(1-E-propenylamino) uridine. In some embodiments, an oligonucleotide as described herein comprises a modified cytosine. Example nucleobases and nucleosides having a modified cytosine include 5-aza-cytidine, 6-aza-cytidine, pseudoisocytidine, 4-methyl-cytidine (m3C), N4-acetyl-cytidine (ac4C), 5-formyl-cytidine (f5C), N4-methyl-cytidine (m4C), 5-methyl-cytidine (m5C), 5-halo-cytidine (e.g., 5-iodo-cytidine), 5-hydroxymethyl-cytidine (hm5C), 1-methyl-pseudoisocytidine, pyrrolo-cytidine, pyrrolo-pseudoisocytidine, 2-thio-cytidine (s2C), 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, lysidine (k2C), a-thio-cytidine, 21-O-methyl-cytidine (Cm), 5,2′-O-dimethyl-cytidine (m5Cm), N4-acetyl-2′-O-methyl-cytidine (ac4Cm), N4,2′-O-dimethyl-cytidine (m4Cm), 5-formyl-2′-O-methyl-cytidine (f5Cm), N4,N4,2′-O-trimethyl-cytidine (m4 2 Cm), 1-thio-cytidine, 2′-F-aracytidine, 2′-F-cytidine, and 21-OH-aracytidine.

When nucleic acids sequences are referred to herein, the current disclosure is generally understood to include nucleic acid sequences with at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%, or 100% complementarity or sequence identity.

By “hybridize” it is generally meant that a nucleic acid (e.g. RNA, DNA) contains a sequence of nucleotides that enables it to non-covalently bind, e.g. form Watson-Crick base pairs or G/U base pairs, “to another nucleic acid in a sequence-specific, antiparallel, manner (e.g., a nucleic acid specifically binds to a complementary nucleic acid) under the appropriate in vitro or in vivo conditions of temperature and solution ionic strength. Standard Watson-Crick base-pairing includes: adenine/adenosine) (A) pairing with thymidine/thymidine (T), A pairing with uracil/uridine (U), and guanine/guanosine) (G) pairing with cytosine/cytidine (C). Inosine (I) bases pair with cytosine/cytidine. In addition, for hybridization between two RNA molecules (e.g., dsRNA), and for hybridization of a DNA molecule with an RNA molecule (e.g., when a DNA splint oligo base pairs with an mRNA segment, etc.): G can also base pair with U. For example, G/U base-pairing is partially responsible for the degeneracy (e.g., redundancy) of the genetic code in the context of tRNA anti-codon base-pairing with codons in mRNA. Thus, in the context of this disclosure, a G (e.g., of a protein-binding segment (e.g., dsRNA duplex) of a guide RNA molecule; of a target nucleic acid (e.g., target DNA or RNA) base pairing with a sensor RNA) is generally considered complementary to both a U and to C. For example, when a G/U base-pair can be made at a given nucleotide position of a protein-binding segment (e.g., dsRNA duplex) of a sensor RNA molecule, the position is not generally considered to be non-complementary, but is instead generally considered to be complementary. Pseudouridine (pseudo-U, ‘I’) denotes an isomer of uridine, where the uracil nucleobase is attached through a carbon-carbon linkage to the sugar. The pseudouridine is in some cases modified. In some cases the pseudouridine modification is methylation, e.g., at the Ni position, forming N1-methylpseudouridine. Pseudouridine and its modifications base pair like uridine. When U is shown in a sequence, e.g. UAG, the U may be a pseudouridine. When a T is shown in a sequence, it is generally meant that the RNA encoded by that sequences contains a U, a pseudouridine, or a modified pseudouridine. The DNA that encodes a U or pseudouridine contains T in place of U or pseudouridine. “Mismatched” generally refers to a base that is opposite a non-complementary base in an otherwise double-stranded structure (e.g., a C: A mismatch), or that a base is opposite no bases (e.g., a base is in a loop structure).

Hybridization generally requires that the two nucleic acids contain complementary sequences, although mismatches between bases are possible. The conditions appropriate for hybridization between two nucleic acids depend on the length of the nucleic acids and the degree of complementarity. The greater the degree of complementarity between two nucleotide sequences, the greater the value of the melting temperature (Tm) for hybrids of nucleic acids having those sequences. In some cases, the length for a nucleic acid hybridizing to another nucleic acid is 8 nucleotides or more (e.g., 10 nucleotides or more, 12 nucleotides or more, 15 nucleotides or more, 20 nucleotides or more, 22 nucleotides or more, 25 nucleotides or more, or 40 nucleotides or more). It is generally understood that the sequence of a nucleic acid need not be 100% complementary to that of its target nucleic acid to hybridize. Moreover, a polynucleotide may hybridize over one or more segments such that intervening or adjacent segments are not involved in the hybridization event (e.g., a loop structure or hairpin structure, a ‘bulge’, and the like). A polynucleotide can include 60% or more, 65% or more, 70% or more, 75% or more, 80% or more, 85% or more, 90% or more, 95% or more, 98% or more, 99% or more, 99.5% or more, or 100% sequence complementarity to a target region within the target nucleic acid sequence to which it will hybridize. For example, an antisense nucleic acid in which 18 of 20 nucleotides of the antisense compound are complementary to a target region (e.g. is capable of specific hybridization) can correspond to 90 percent complementarity. The remaining noncomplementary nucleotides may be clustered or interspersed with complementary nucleotides and need not be contiguous to each other or to complementary nucleotides. Percent complementarity between particular stretches of nucleic acid sequences within nucleic acids can be determined using any convenient method. Example methods include BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), e.g., using default settings, which uses the algorithm of Smith and Waterman (Adv. Appl. Math., 1981, 2, 482-489).

A method of treating a subject may comprise or suspected of may comprise hypertension or hyperlipidemia, may comprise administering a nucleic acid molecule or a salt thereof configured to hybridize to a PDZK1 sequence to subject.

The nucleic acid molecule or salt thereof may comprise a short interfering RNA (siRNA) molecule, a short hairpin RNA (shRNA) molecule, a microRNA adapted shRNA (shRNAmiR) molecule, a microRNA (miRNA molecule), or an antisense oligonucleotide (ASO), or any combination thereof.

The nucleic acid molecule or salt thereof may comprise a siRNA molecule. The siRNA molecule may comprise a duplex region of about 18 to about 27 base pairs.

The PDZK1 sequence may be a PDZK1 mRNA sequence.

The nucleic acid molecule may be not encapsulated in an exosome.

The nucleic acid molecule may comprise a sequence may comprise at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%, or 100% complementarity or identity to at least 18-27 consecutive nucleotides of SEQ ID NO: 29.

The nucleic acid molecule may comprise a sequence may comprise at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%, or 100% sequence identity to any one of SEQ ID NOs: 1-28.

The subject has or may be suspected of may comprise hyperlipidemia. The hyperlipidemia may comprise hypercholesterolemia.

The hypercholesterolemia may comprise a serum LDL level of greater than about 125 mg/dL.

The hypercholesterolemia may comprise a serum LDL: serum HDL ratio of less than about 3.5:1.

The subject has or may be suspected of may comprise hyperlipidemia. The hyperlipidemia may comprise glyceridemia.

The glyceridemia may comprise hypertriglyceridemia.

The hypertriglyceridemia may comprise a serum triglyceride level of greater than about 150 mg/dL.

The subject has or may be suspected of may comprise hypertension.

A method of treating a subject may comprise or suspected of may comprise glyceridemia, may comprise administering a nucleic acid molecule or a salt thereof to subject. The nucleic acid molecule may comprise a sequence may comprise at least 80% sequence identity to any one of SEQ ID NOs: 1-28, or a variant thereof.

The glyceridemia may comprise hypertriglyceridemia.

A method of treating a subject may comprise or suspected of may comprise hyperlipidemia, may comprise administering a nucleic acid molecule or a salt thereof to subject. The nucleic acid molecule may comprise a sequence may comprise at least 80% sequence identity to any one of SEQ ID NOs: 1, 2, 7, or 10-12, 14-28, or a variant thereof

The hyperlipidemia may comprise hypercholesterolemia.

The hyperlipidemia may comprise hypertriglyceridemia.

A method of treating a subject may comprise or suspected of may comprise hypertension, may comprise administering a nucleic acid molecule or a salt thereof to subject. The nucleic acid molecule may comprise a sequence may comprise at least 80% sequence identity to any one of SEQ ID NOs: 1, 2, 7, or 10-28, or a variant thereof

The nucleic acid molecule may be an siRNA molecule may comprise a duplex region.

The duplex region may be 18-27 base pairs in length.

An siRNA molecule or a salt thereof may comprise a duplex region 18-27 base pairs in length. The duplex region may comprise a sequence may comprise at least 80% sequence identity to any one of SEQ ID NOs: 2, 15-22, 24-26, or 28.

An siRNA molecule, or a salt thereof may comprise a duplex region 18-27 base pairs in length. The duplex region may comprise a sequence may comprise at least 90% sequence identity to any one of SEQ ID NOs: 2, 7, 11, 15-22, or 23-28, or a variant thereof

An siRNA molecule or a salt thereof may comprise a duplex region 18-27 base pairs in length. The duplex region may comprise a sequence may comprise at least 91% sequence identity to any one of SEQ ID NOs: 2, 3, 4, 7, 8, 9, 11, 15-22, or 23-28, or a variant thereof.

An siRNA molecule or a salt thereof may comprise a duplex region 18-27 base pairs in length. The duplex region may comprise a sequence may comprise at least 96% sequence identity to any one of SEQ ID NOs: 2, 3, 4, 7, 8, 9, 11, 12, 15-22, or 23-28, or a variant thereof.

The duplex region may be 20-27 base pairs in length.

An siRNA molecule or a salt thereof may comprise a duplex region 20-27 base pairs in length. The duplex region may comprise a sequence may comprise at least 80% sequence identity to any one of SEQ ID NOs: 1, 2, 3, 4, 8, 9, or 15-22, or 23-28, or a variant thereof.

The siRNA molecule may comprise one or more non-natural RNA nucleotide or one or more non-natural linkage.

The siRNA molecule may comprise one or more non-natural RNA nucleotide. The one or more non-natural RNA nucleotide may comprise 2′-fluoro, 2′-O-methyl, 2¹-O-methyl, 2¹-O-methoxy-ethyl, 2¹-O-methoxy-ethoxy, 5′-methyl, SNA, hGNA, hhGNA, mGNA, TNA, h′GNA, a locked nucleic acid (LNA), or any combination thereof.

The siRNA molecule may comprise one or more non-natural linkage. The one or more non-natural linkage may comprise a phosphorothioate or methylphosphonate linkage.

TABLE 2
Sequences according to methods, compositions, and systems described herein.

| SEQ | | |
|---|---|---|
| ID | | |
| NO: | Description | Sequence |
| 1. | siRNA/shRNA | GCTTCTTCCTGCGAATTGAGA |
| | targeting | |
| | sequence | |
| 2. | siRNA/shRNA | GAAGTCAGGAAGCCGTGTCAT |
| | targeting | |
| | sequence | |
| 3. | siRNA/shRNA | GAGCAGAAGATACAATTCAAA |
| | targeting | |
| | sequence | |
| 4. | siRNA/shRNA | GGTCAAATCATCAAGGACATA |
| | targeting | |
| | sequence | |
| 5. | siRNA/shRNA | GAAGAACAATGATCTGGTAGT |
| | targeting | |
| | sequence | |
| 6. | siRNA/shRNA | GCAGGCTGGCTAAAGGTGAAA |
| | targeting | |
| | sequence | |
| 7. | siRNA/shRNA | GGCTCATTCATCAAAGAGGTA |
| | targeting | |
| | sequence | |
| 8. | siRNA/shRNA | GTGGAAAGAAGGCCTATGATT |
| | targeting | |
| | sequence | |
| 9. | siRNA/shRNA | GGAAAGAAGGCCTATGATTAT |
| | targeting | |
| | sequence | |
| 10. | siRNA/shRNA | GGCTATTGCAGACGGCTAATT |
| | targeting | |
| | sequence | |
| 11. | siRNA/shRNA | GCTATTGCAGACGGCTAATTT |
| | targeting | |
| | sequence | |
| 12. | siRNA/shRNA | GCTTAACTTAGGAAGAGATAA |
| | targeting | |
| | sequence | |
| 13. | siRNA/shRNA | GGAAGAGATAAGGCAAGAGCT |
| | targeting | |
| | sequence | |
| 14. | siRNA/shRNA | GAGATAAGGCAAGAGCTAGAT |
| | targeting | |
| | sequence | |
| 15. | siRNA/shRNA | GTAGGTCGCCATCTTGCTATA |
| | targeting | |
| | sequence | |
| 16. | siRNA/shRNA | GATGGTCAGATCCGCAGAGTA |
| | targeting | |
| | sequence | |
| 17. | siRNA/shRNA | GGAACAACAGTATAGAACATA |
| | targeting | |
| | sequence | |
| 18. | siRNA/shRNA | GACCTAATCATAGCGATAGAA |
| | targeting | |
| | sequence | |
| 19. | siRNA/shRNA | GGATTAAGCGATGACGTATAA |
| | targeting | |
| | sequence | |
| 20. | siRNA/shRNA | GGTGCGGGACGACAATGAATA |
| | targeting | |
| | sequence | |
| 21. | siRNA/shRNA | GGATATGGTCGCCAACTTAAA |
| | targeting | |
| | sequence | |
| 22. | siRNA/shRNA | GTGAAGTAGCGAAGTCATGTA |
| | targeting | |
| | sequence | |
| 23. | siRNA/shRNA | GGAATGATTAAGGTCGATACA |
| | targeting | |
| | sequence | |
| 24. | siRNA/shRNA | GTAAGTCGTCACGGTAGTACT |
| | targeting | |
| | sequence | |
| 25. | siRNA/shRNA | GACTTGGCACTTAGCGTATAT |
| | targeting | |
| | sequence | |
| 26. | siRNA/shRNA | GAACAAGAACGATTAGTTAGT |
| | targeting | |
| | sequence | |
| 27. | siRNA/shRNA | GAAGAGCAGACGTAGGAGTAA |
| | targeting | |
| | sequence | |
| 28. | siRNA/shRNA | GACGACAAAGTAGGGAATGAT |
| | targeting | |
| | sequence | |
| 29. | PDSK1 | GAATTCCGGGCAGCTCCTCTTCCATCTCCAGAAATGACCTCCAC |
| | mRNA | CTTCAACCCCCGAGAATGTAAACTGTCCAAGCAAGAAGGGCAA |
| | (genbank | AACTATGGCTTCTTCCTGCGAATTGAGAAGGACACCGAGGGCCA |
| | AF012281.1) | CCTGGTCCGGGTGGTTGAGAAGTGTAGCCCAGCAGAGAAGGCT |
| | | GGCCTTCAAGATGGAGACAGAGTTCTTAGGATCAATGGTGTCTT |
| | | TGTGGACAAAGAAGAACATATGCAGGTTGTGGATCTGGTCAGA |
| | | AAGAGTGGGAATTCAGTGACTTTACTAGTTCTGGATGGGGATTC |
| | | CTATGAGAAAGCAGTGAAAACACGGGTGGACTTGAAAGAGTTG |
| | | GGTCAAAGTCAGAAGGAGCAAGGTTTGAGTGATAATATACTTTC |
| | | CCCTGTGATGAATGGAGGTGTGCAAACTTGGACCCAGCCCCGGC |
| | | TCTGCTATCTCGTGAAGGAAGGAGGCAGCTATGGCTTCTCTCTG |
| | | AAAACTGTCCAAGGTAAAAAGGGGGTGTACATGACTGATATTA |
| | | CACCTCAAGGTGTGGCTATGAGAGCTGGAGTTCTGGCTGATGAT |
| | | CACTTGATTGAAGTGAATGGAGAGAATGTAGAGGATGCCAGCC |
| | | ATGAGAAAGTGGTTGAAAAGGTGAAGAAGTCAGGAAGCCGTGT |
| | | CATGTTCCTGCTGGTGGACAAAGAAACTGACAAGCGTCATGTTG |
| | | AGCAGAAGATACAATTCAAAAGAGAAACAGCCAGTTTGAAACT |
| | | GTTACCCCACCAGCCCCGAATTGTGGAGATGAAGAAAGGAAGC |
| | | AATGGCTATGGTTTCTATCTGAGGGCAGGCTCAGAACAGAAAG |
| | | GTCAAATCATCAAGGACATAGATTCTGGAAGTCCAGCAGAGGA |
| | | GGCTGGCTTGAAGAACAATGATCTGGTAGTTGCTGTCAACGGCG |
| | | AGTCTGTGGAAACCCTGGATCATGACAGTGTGGTAGAAATGATT |
| | | AGAAAGGGTGGAGATCAGACTTCACTGTTGGTGGTAGACAAAG |
| | | AGACGGACAACATGTACAGACTGGCTCATTTTTCTCCATTTCTCT |
| | | ACTATCAAAGTCAAGAACTGCCCAATGGCTCTGTCAAGGAGGCT |
| | | CCAGCTCCTACTCCCACTTCTCTGGAAGTCTCAAGTCCACCAGA |
| | | TACTACAGAGGAAGTAGATCATAAGCCTAAACTCTGCAGGCTG |
| | | GCTAAAGGTGAAAATGGCTATGGCTTTCACTTAAATGCGATTCG |
| | | GGGTCTGCCAGGCTCATTCATCAAAGAGGTACAGAAGGGCGGT |
| | | CCTGCTGACTTGGCTGGGCTAGAGGATGAGGATGTCATCATTGA |
| | | AGTGAATGGGGTGAATGTGCTAGATGAACCCTATGAGAAGGTG |
| | | GTGGATAGAATCCAGAGCAGTGGGAAGAATGTCACACTTCTAG |
| | | TCTGTGGAAAGAAGGCCTATGATTATTTCCAAGCTAAGAAAATC |
| | | CCTATTGTTTCCTCCCTGGCTGATCCACTTGACACCCCTCCAGAT |
| | | TCTAAAGAAGGAATAGTGGTGGAGTCAAACCATGACTCGCACA |
| | | TGGCAAAAGAACGGGCCCACAGTACAGCCTCACATTCTTCTTCC |
| | | AATTCTGAAGATACAGAGATGTGATGAAAACAAGTAATAGCTTT |
| | | GGCTGTTTATTTGATAGCTGTTTCTGGGTATTTAATAGGAATCCT |
| | | TTCTCAAGGAATGAGTTGTGACCTGTTTACTGTCTCTTTAGAAGA |
| | | AAAACTCCACTGGAAACCATTCACCATGTGTGACTGTCTTCTGT |
| | | TATCATTTGTCTTACAGGCGGCTATTGCAGACGGCTAATTTATGC |
| | | TTAACTTAGGAAGAGATAAGGCAAGAGCTAGATTTTTTTCATGT |
| | | GATCTTTTCCAAGCTTCAACTTAACTTAACTACATTTCTCTGTAT |
| | | GATGATGTCTCTTACTTCTACAGGTTCCTTGAGCACCAAAGATG |
| | | ATTCATAACTCTGTATAGGTGACAGCTGCTTATAAAAGCATCTT |
| | | AGCAGATAAGCCTATTAAAATTGTGCTTTTGTAACAATGTTGTG |
| | | GTTGCTAGAATAAATACCATGAACCCG |

### EXAMPLES

The following examples are provided to further illustrate some embodiments of the present disclosure, but are not intended to limit the scope of the disclosure; it will be understood by their exemplary nature that other procedures, methodologies, or techniques may alternatively be used.

### Example 1: Variant Assessment and Analysis

### Methods

### Recruitment and Clinical Environment

The FH Mexican Registry was utilized, for which design and rationale has been described (see e.g. Mehta et al. Atherosclerosis, vol. 277, pp. 517-523, October 2018, doi: 10.1016/j.atherosclerosis.2018.06.007, which is incorporated by reference herein in its entirety). This research protocol was approved by the Institutional Research and Ethics Committees (Ref 2571). Patients identified with baseline LDL-C levels >190 mg/dL (adults) or >160 mg/dL (children) were evaluated in accordance with the Dutch Lipid Clinic Network criteria 19. Only those cases that have a definite or probable diagnosis were included in the database. Decision to participate in the study and informed consent form (ICF) were obtained from each subject. Within the web-based registry information was captured from subjects with heterozygous/homozygous familial hypercholesterolemia (FH) clinical diagnosis.

### Sequencing Twist Kit (WES and DP)

In the laboratory facilities of Galatea bio, 402 samples from 169 families were sequenced using whole exome sequencing (WES), whole genome sequencing, and a custom low-pass genome sequencing approach. Combined, these samples were interrogated for both common and rare genetic variants associated with FH. WES sequencing libraries were prepared using the Twist Exome 2.0/Twist Library Preparation EF Kit 2.0 with Twist SNP Density Panel spiked in. The hybridized fragments were then sequenced as 2×150 bp reads on an Illumina NovaSeq 6000 instrument (Illumina, San Diego, CA, USA).

### SNV Analysis

Initially, analysis focused on three genes documented to cause FH: LDLR, APOB and PCSK9. Variants were annotated using Variant Effect Predictor (VEP)(see e.g. McLaren et al Genome Biol, vol. 17, no. 1, p. 122, June 2016, doi: 10.1186/s13059-016-0974-4, which is incorporated by reference herein in its entirety) and filtered for a population-based gnomAD allele frequency less than 1% (https://gnomad.broadinstitute.org/). The ClinVar database was used to classify the variants in terms of pathogenicity. Next, non-classical genes documented to cause FH (APOE, LIPA, ABCG5, ABCG8) were examined. To discover potentially new genes for FH, rare variants in 47 genes in the core cholesterol pathway (KEGG pathway: hsa048979) were searched for and interactors (55 genes) acquired from the Intact database (see e.g. Hermjakob et al. Nucleic Acids Res, vol. 42, no. Database issue, pp. D452-D455, January 2004, doi: 10.1093/nar/gkh052, which is incorporated by reference herein in its entirety) were added.

### Cnv Calling.

CNV Calling was performed using two algorithms designed for WES data, CODEX (see e.g. Jiang et al. Nucleic Acids Res, vol. 43, no. 6, p. e39, March 2015, doi: 10.1093/nar/gku1363, which is incorporated by reference herein in its entirety) and ExomeDepth (see e.g. Plagnol et al. Bioinformatics, vol. 28, no. 21, pp. 2747-2754 November 2012, doi: 10.1093/bioinformatics/bts526, which is incorporated by reference herein in its entirety), using the recommended default settings of each program. Briefly, to generate the normalized coverage metrics for each exon, the bed files of the FH genes were supplied as inputs and the bam files were processed in batches according to each WES run. CODEX normalizes the depth of coverage by applying a Poisson latent factor model that removes bias due to GC content, exon capture and amplification efficiency, and latent systemic artifacts. Five Poisson latent factors were included in the normalization model for this dataset. For each sample, ExomeDepth builds a reference set that includes samples with similar read count distribution and then performs CNV calling. CNVs that were detected by both programs were prioritized. CNVs were called for all documented FH genes, as well as for new candidate genes involved in or interacting with key effectors of the cholesterol pathway.

### CNV Validation Using Taqman Assay

To validate the CNV, custom TaqMan probes and primers targeting the genomic region of PDZK1 were designed. TaqMan assays were then conducted to assess the presence of the CNV.

### PheWas Studies

The pheWeb database (See e.g. Taliun et al. Nat Genet, vol. 52, no. 6, pp. 550-552, June 2020, doi: 10.1038/s41588-020-0622-5, which is incorporated by reference in its entirety herein) was utilized to retrieve pheWas results for variants in PDZK1. This database contains the results of pheWas studies conducted in large datasets, including the UKBiobank, the Michigan Genomics Initiative and the FinMetSeq study.

### Results

Discovery of a new gene responsible for autosomal dominant FH.

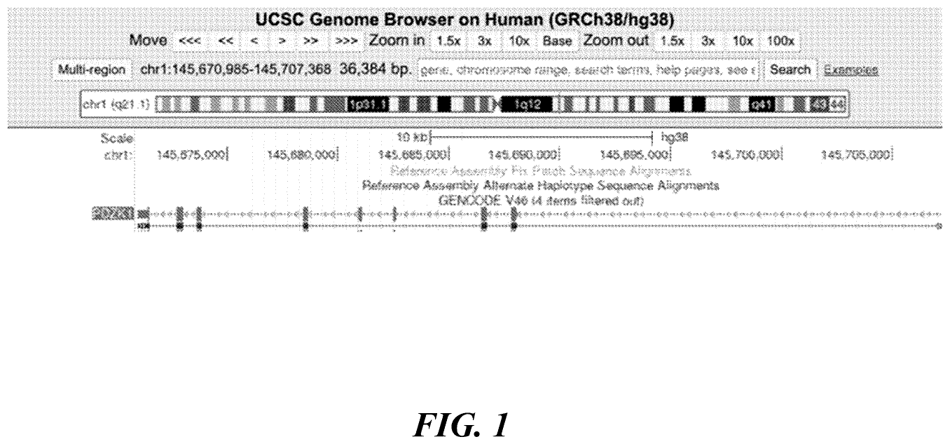

Following CNV analysis, a whole-gene duplication of PDZK1 was identified in two unrelated FH patients (FIG. 1). PDZK1, which encodes a scaffolding protein from the PDZ domain-containing family, was included in the list of candidate genes for FH because it can interact with and stabilize the scavenger receptor class B type I (SR-BI), which in turn is involved in high-density lipoprotein (HDL) metabolism. Specifically, SR-BI is documented as facilitating the uptake of HDL cholesterol by the liver, a critical process for maintaining cholesterol homeostasis. Both patients harboring the whole-gene duplication were male and, aside from a confirmed diagnosis of FH, one patient also had associated detailed clinical records. At the age of 26, this individual with detailed clinical records presented with a total cholesterol level of 456 mg/dL, LDL cholesterol of 474 mg/dL, and HDL cholesterol of 44.8 mg/dL.

Table 1 below presents the CNV metrics generated by the two programs, CODEX and Exomedepth. In particular, Ratio Metrics such as LRatio for CODEX highlight the deviation of observed coverage from expected levels, while the Reads Ratio for Exomedepth quantifies the proportion of observed reads compared to expected reads. Coverage Metrics from CODEX include both raw and normalized coverage, while Exomedepth provides the expected and observed read counts to assess CNV presence and size.

Notably, this duplication was found only in 5/200,000 genomes from the All of Us Project (https://alofiis.nih.gov/), thus showing a significant enrichment (p<10-4) in our FH cohort.

TABLE 1
CNV metrics generated by the two programs, CODEX and Exomedepth, indicating
the presence of a duplication of the entire PDZK1 gene in two patients.
CODEX METRICS

| ID | Coordinates | length_kb | raw_cov | norm_cov | copy_no | lratio | mBIC |
|---|---|---|---|---|---|---|---|
| BYMD-274- | chrl: | 24603.65 | 4563 | 3167 | 3 | 266.561 | 240.591 |
| 153- 001- | | | | | | | |
| BYMD-197- | chrl: | 1904.378 | 7212 | 4639 | 3 | 604.594 | 1206.458 |
| 123- 001- | | | | | | | |

EXOMEDEPTH METRICS

| | | | Reads | Reads | Reads |
|---|---|---|---|---|---|
| ID | Coordinates | BF | expected | observed | ratio |
| BYMD-274- | chrl: | 78.5 | 2410 | 3597 | 1.49 |
| 153- 001- | | | | | |
| BYMD-197- | chrl: | 130 | 4066 | 5918 | 1.46 |
| 123- 001- | | | | | |

FIG. 1 shows a schematic representation of the duplication (chrl: 145670985-145707368) detected in two unrelated patients projected on the UCSC Genome Browser (https://genome.ucsc.edu/).

Next, whether or not any association of LDL metabolism-relevant phenotypes with PDZK1 could be detected in the general population was determined. For this purpose, PheWas analyses were performed across several large cohort datasets.

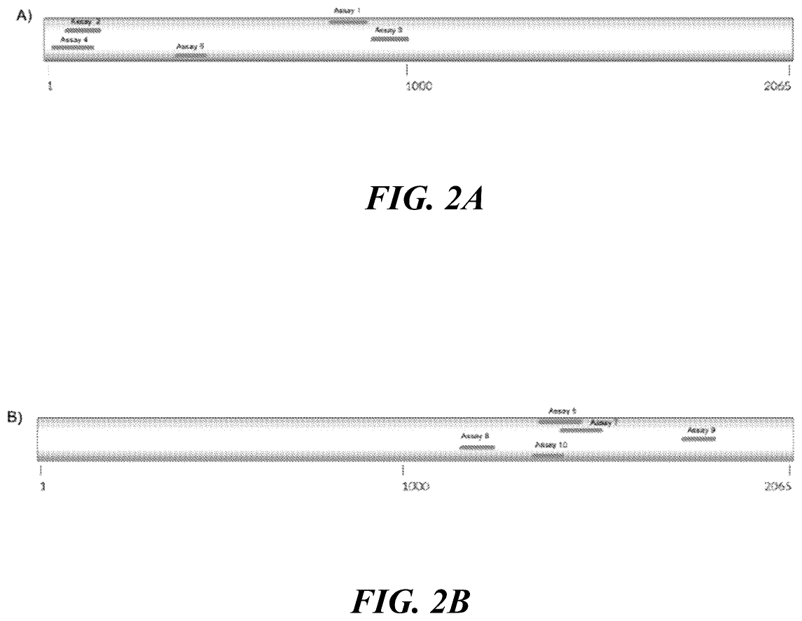

FIGS. 2A-2B show assay results for rs2976384 for Assay 1 through Assay 10.

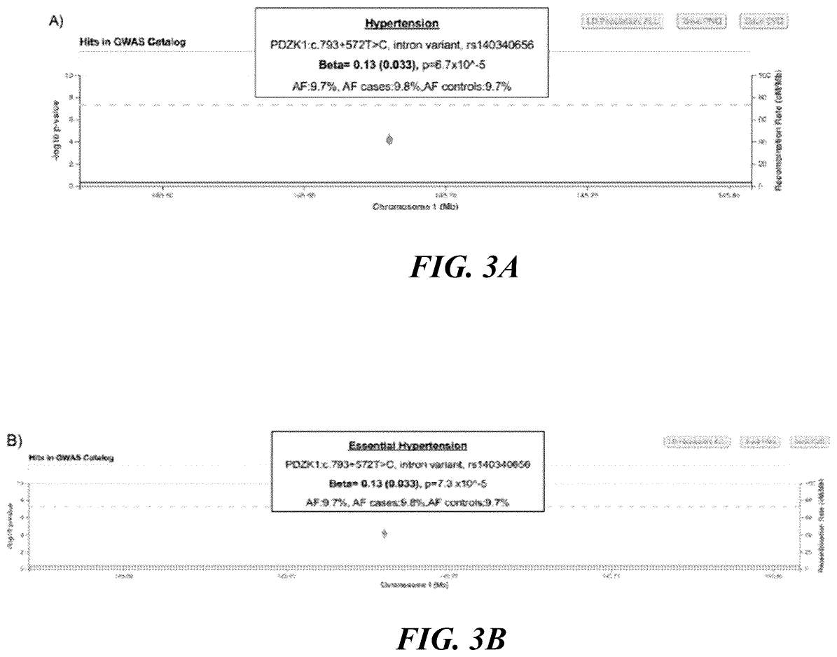

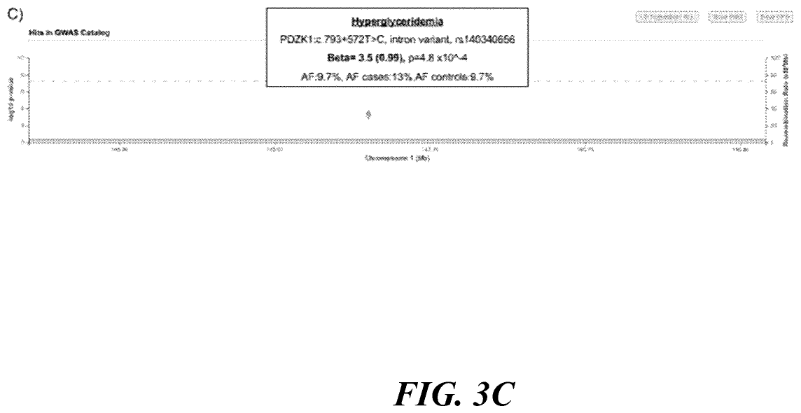

In the UKBiobank the intron variant c. 793+572T>C (rs140340656) in the PDZK1 locus was found to be associated with higher risk of hypertension (Beta=0.13, SE=0.033, p=6.7×10-5); essential hypertension (Beta=0.13, SE=0.033, p=7.3*10-5); and hyperglyceridemia (Beta=3.5, SE=0.99, p=4.8×10-4) (FIGS. 3A-3C).

FIGS. 3A-3C shows a phenome-wide view for variant rs2976384 showing phenome-wide significant association with traits from the UK Biobank including hypertension (A), essential hypertension (B) and hyperglyceridemia (C).

Furthermore, in the FinMetSeq cohort, a different intronic variant, c.211-69A>G (rs1553701870) of PDZK1 was found to be associated with significantly lower levels of saturated fatty acids (Beta=−1.7, SE=0.45, p=1.4×10-4) and LDL cholesterol (Beta=−1.4, SE=0.38, p=4.3×10-4). Finally, a third intronic variant, c.416-195A>G (r51649823291) was found to be associated with reduced levels of ApoB (Beta=−1.1, SE=0.33, p=5.6×10-4) within the same cohort (FIGS. 3A-3C).

Phenome-wide view (PheWAS) for variants rs1553701870 and rs 1649823291 were analyzed and showed phenome-wide significant association with traits from the FinMetSeq study including saturated fatty acids (A), LDL cholesterol (B) and APOB levels (C).

### Suppression of PDZK1 for the Treatment of Hypercholesterolemia and Related Disorders

Based on these results, suppressing PDZK1 expression has the potential of leading to favorable outcomes in patients with familial hypercholesterolemia (FH) and related disorders, such as hypertension and stroke. To explore this, siRNA oligo targeting sequences specifically targeting human PDZK1 mRNA were designed. Here, we report nucleic acid targeting sequences (e.g. siRNA sequences) designed for the therapeutic silencing of PDZK1 expression, reduction of translation, or both (see Table 2).

Consistent with these findings, studies have observed elevated HDL levels in PDZKI knockout mouse models, a phenotype aligned with the findings in our duplication patients. Without wishing to bound by theory, it is possible that PDZK1 is necessary for full SR-BI HDL receptor expression, as it has been observed that suppression of PDZK1 can lead to a tissue-specific, significant reduction in SR-BI expression (e.g. in the liver and proximal intestine rather than other steroidogenic organs such as adrenal glands, ovaries, and testes).
The genetic data both in rare FH cases and in the general population supplied herein suggests that targeted suppression of PDZK1 will yield beneficial outcomes for regulating LDL levels and thus prevent some of the serious complications secondary to hypercholesterolemia, including heart disease and stroke. Given the documented biology of LDL regulation in the liver and the expression of PDZK1 in that organ, the suppression of expression or translation of this molecule by the nucleic acid molecules shown in Table 2 is a likely effective agent for the therapy of hyperlipidemia and the prevention of severe healthcare outcomes.

### Computing Systems

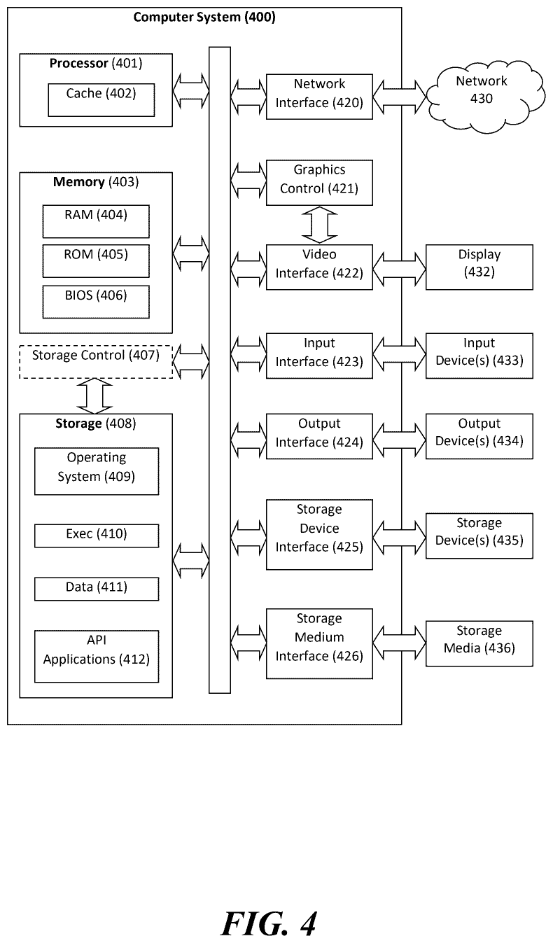

Referring to FIG. 4, a block diagram is shown depicting an exemplary machine that includes a computer system 400 (e.g., a processing or computing system) within which a set of instructions can execute for causing a device to perform or execute any one or more of the aspects and/or methodologies for static code scheduling of the present disclosure. The components in FIG. 4 are examples only and do not limit the scope of use or functionality of any hardware, software, embedded logic component, or a combination of two or more such components implementing particular embodiments.

Computer system 400 may include one or more processors 401, a memory 403, and a storage 407 that communicate with each other, and with other components, via a bus 440. The bus 440 may also link a display 432, one or more input devices 433 (which may, for example, include a keypad, a keyboard, a mouse, a stylus, etc.), one or more output devices 434, one or more storage devices 435, and various tangible storage media 436. All of these elements may interface directly or via one or more interfaces or adaptors to the bus 440. For instance, the various tangible storage media 436 can interface with the bus 440 via storage medium interface 426. Computer system 400 may have any suitable physical form, including but not limited to one or more integrated circuits (ICs), printed circuit boards (PCBs), mobile handheld devices (such as mobile telephones or PDAs), laptop or notebook computers, distributed computer systems, computing grids, or servers.

Computer system 400 includes one or more processor(s) 401 (e.g., central processing units (CPUs) or general-purpose graphics processing units (GPGPUs)) that carry out functions. Processor(s) 401 optionally contains a cache memory unit 402 for temporary local storage of instructions, data, or computer addresses. Processor(s) 401 are configured to assist in execution of computer readable instructions. Computer system 400 may provide functionality for the components depicted in FIG. 4 as a result of the processor(s) 401 executing non-transitory, processor-executable instructions embodied in one or more tangible computer-readable storage media, such as memory 403, storage 408, storage devices 435, and/or storage medium 436. The computer-readable media may store software that implements particular embodiments, and processor(s) 401 may execute the software. Memory 403 may read the software from one or more other computer-readable media (such as mass storage device(s) 435, 436) or from one or more other sources through a suitable interface, such as network interface 420. The software may cause processor(s) 401 to carry out one or more processes or one or more operations of one or more processes described or illustrated herein. Carrying out such processes or operations may include defining data structures stored in memory 403 and modifying the data structures as directed by the software.

The memory 403 may include various components (e.g., machine readable media) including, but not limited to, a random access memory component (e.g., RAM 404) (e.g., static RAM (SRAM), dynamic RAM (DRAM), ferroelectric random access memory (FRAM), phase-change random access memory (PRAM), etc.), a read-only memory component (e.g., ROM 405), and any combinations thereof. ROM 405 may act to communicate data and instructions unidirectionally to processor(s) 401, and RAM 404 may act to communicate data and instructions bidirectionally with processor(s) 401. ROM 405 and RAM 404 may include any suitable tangible computer-readable media described below. In one example, a basic input/output system 406 (BIOS), including basic routines that help to transfer information between elements within computer system 400, such as during start-up, may be stored in the memory 403.

Fixed storage 408 is connected bidirectionally to processor(s) 401, optionally through storage control unit 407. Fixed storage 407 provides additional data storage capacity and may also include any suitable tangible computer-readable media described herein. Storage 408 may be used to store operating system 409, executable(s) 410, data 411, applications 412 (application programs), and the like. Storage 408 can also include an optical disk drive, a solid-state memory device (e.g., flash-based systems), or a combination of any of the above. Information in storage 408 may, in appropriate cases, be incorporated as virtual memory in memory 403.

In one example, storage device(s) 435 may be removably interfaced with computer system 400 (e.g., via an external port connector (not shown)) via a storage device interface 425. Particularly, storage device(s) 435 and an associated machine-readable medium may provide non-volatile and/or volatile storage of machine-readable instructions, data structures, program modules, and/or other data for the computer system 400. In one example, software may reside, completely or partially, within a machine-readable medium on storage device(s) 435. In another example, software may reside, completely or partially, within processor(s) 401.

Bus 440 connects a wide variety of subsystems. Herein, reference to a bus may encompass one or more digital signal lines serving a common function, where appropriate. Bus 440 may be any of several types of bus structures including, but not limited to, a memory bus, a memory controller, a peripheral bus, a local bus, and any combinations thereof, using any of a variety of bus architectures. As an example and not by way of limitation, such architectures include an Industry Standard Architecture (ISA) bus, an Enhanced ISA (EISA) bus, a Micro Channel Architecture (MCA) bus, a Video Electronics Standards Association local bus (VLB), a Peripheral Component Interconnect (PCI) bus, a PCI-Express (PCI-X) bus, an Accelerated Graphics Port (AGP) bus, HyperTransport (HTX) bus, serial advanced technology attachment (SATA) bus, and any combinations thereof.

Computer system 400 may also include an input device 433. In one example, a user of computer system 400 may enter commands and/or other information into computer system 400 via input device(s) 433. Examples of an input device(s) 433 include, but are not limited to, an alpha-numeric input device (e.g., a keyboard), a pointing device (e.g., a mouse or touchpad), a touchpad, a touch screen, a multi-touch screen, a joystick, a stylus, a gamepad, an audio input device (e.g., a microphone, a voice response system, etc.), an optical scanner, a video or still image capture device (e.g., a camera), and any combinations thereof. In some embodiments, the input device is a Kinect, Leap Motion, or the like. Input device(s) 433 may be interfaced to bus 440 via any of a variety of input interfaces 423 (e.g., input interface 423) including, but not limited to, serial, parallel, game port, USB, FIREWIRE, THUNDERBOLT, or any combination of the above.

In particular embodiments, when computer system 400 is connected to network 430, computer system 400 may communicate with other devices, specifically mobile devices and enterprise systems, distributed computing systems, cloud storage systems, cloud computing systems, and the like, connected to network 430. Communications to and from computer system 400 may be sent through network interface 420. For example, network interface 420 may receive incoming communications (such as requests or responses from other devices) in the form of one or more packets (such as Internet Protocol (IP) packets) from network 130, and computer system 400 may store the incoming communications in memory 403 for processing. Computer system 400 may similarly store outgoing communications (such as requests or responses to other devices) in the form of one or more packets in memory 403 and communicated to network 430 from network interface 420. Processor(s) 401 may access these communication packets stored in memory 403 for processing.

Examples of the network interface 420 include, but are not limited to, a network interface card, a modem, and any combination thereof. Examples of a network 430 or network segment 430 include, but are not limited to, a distributed computing system, a cloud computing system, a wide area network (WAN) (e.g., the Internet, an enterprise network), a local area network (LAN) (e.g., a network associated with an office, a building, a campus or other relatively small geographic space), a telephone network, a direct connection between two computing devices, a peer-to-peer network, and any combinations thereof. A network, such as network 430, may employ a wired and/or a wireless mode of communication. In general, any network topology may be used.

Information and data can be displayed through a display 432. Examples of a display 432 include, but are not limited to, a cathode ray tube (CRT), a liquid crystal display (LCD), a thin film transistor liquid crystal display (TFT-LCD), an organic liquid crystal display (OLED) such as a passive-matrix OLED (PMOLED) or active-matrix OLED (AMOLED) display, a plasma display, and any combinations thereof. The display 432 can interface to the processor(s) 401, memory 403, and fixed storage 408, as well as other devices, such as input device(s) 433, via the bus 440. The display 432 is linked to the bus 440 via a video interface 422, and transport of data between the display 432 and the bus 440 can be controlled via the graphics control 421. In some embodiments, the display is a video projector. In some embodiments, the display is a head-mounted display (HMD) such as a VR headset. In further embodiments, suitable VR headsets include, by way of non-limiting examples, HTC Vive, Oculus Rift, Samsung Gear VR, Microsoft HoloLens, Razer OSVR, FOVE VR, Zeiss VR One, Avegant Glyph, Freefly VR headset, and the like. In still further embodiments, the display is a combination of devices such as those disclosed herein.

In addition to a display 432, computer system 400 may include one or more other peripheral output devices 434 including, but not limited to, an audio speaker, a printer, a storage device, and any combinations thereof. Such peripheral output devices may be connected to the bus 440 via an output interface 424. Examples of an output interface 424 include, but are not limited to, a serial port, a parallel connection, a USB port, a FIREWIRE port, a THUNDERBOLT port, and any combinations thereof.

In addition or as an alternative, computer system 400 may provide functionality as a result of logic hardwired or otherwise embodied in a circuit, which may operate in place of or together with software to execute one or more processes or one or more operations of one or more processes described or illustrated herein. Reference to software in this disclosure may encompass logic, and reference to logic may encompass software. Moreover, reference to a computer-readable medium may encompass a circuit (such as an IC) storing software for execution, a circuit embodying logic for execution, or both, where appropriate. The present disclosure encompasses any suitable combination of hardware, software, or both.

The various illustrative logical blocks, modules, circuits, and algorithm operations described in connection with the embodiments disclosed herein may be implemented as electronic hardware, computer software, or combinations of both. To clearly illustrate this interchangeability of hardware and software, various illustrative components, blocks, modules, circuits, and operations have been described above generally in terms of their functionality.

The various illustrative logical blocks, modules, and circuits described in connection with the embodiments disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

The operations of a method or algorithm described in connection with the embodiments disclosed herein may be embodied directly in hardware, in a software module executed by one or more processor(s), or in a combination of the two. A software module may reside in RAM memory, flash memory, ROM memory, EPROM memory, EEPROM memory, registers, hard disk, a removable disk, a CD-ROM, or any other form of storage medium. An exemplary storage medium is coupled to the processor such the processor can read information from, and write information to, the storage medium. In the alternative, the storage medium may be integral to the processor. The processor and the storage medium may reside in an ASIC. The ASIC may reside in a user terminal. In the alternative, the processor and the storage medium may reside as discrete components in a user terminal.

In accordance with the description herein, suitable computing devices include, by way of non-limiting examples, cloud computing platforms, distributed computing platforms, server clusters, server computers, desktop computers, laptop computers, notebook computers, sub-notebook computers, netbook computers, and netpad computers.

In some embodiments, the computing device includes an operating system configured to perform executable instructions. The operating system is, for example, software, including programs and data, which manages the device's hardware and provides services for execution of applications. Various suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Various suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Various suitable mobile smartphone operating systems include, by way of non-limiting examples, Nokia® Symbian® OS, AppleR iOS®, Research in Motion® BlackBerry OS®, Google® Android®, Microsoft® Windows Phone® OS, Microsoft® Windows MobileR OS, Linux®, and Palm® WebOS®. Non-transitory Computer Readable Storage Medium

In some embodiments, the platforms, systems, media, and methods disclosed herein include one or more non-transitory computer readable storage media encoded with a program including instructions executable by the operating system of an optionally networked computing device. In further embodiments, a computer readable storage medium is a tangible component of a computing device. In still further embodiments, a computer readable storage medium is optionally removable from a computing device. In some embodiments, a computer readable storage medium includes, by way of non-limiting examples, CD-ROMs, DVDs, flash memory devices, solid state memory, magnetic disk drives, magnetic tape drives, optical disk drives, distributed computing systems including cloud computing systems and services, and the like. In some cases, the program and instructions are permanently, substantially permanently, semi-permanently, or non-transitorily encoded on the media.

### Computer Programs

In some embodiments, the platforms, systems, media, and methods disclosed herein include at least one computer program, or use of the same. A computer program includes a sequence of instructions, executable by one or more processor(s) of the computing device's CPU, written to perform a specified task. Computer readable instructions may be implemented as program modules, such as functions, objects, Application Programming Interfaces (APIs), computing data structures, and the like, which perform particular tasks or implement particular abstract data types. A computer program may be written in various versions of various languages.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Software Modules

In some embodiments, the platforms, systems, media, and methods disclosed herein include software, server, and/or database modules, or use of the same. In view of the disclosure provided herein, software modules are created by various techniques using various machines, software, and languages. The software modules disclosed herein are implemented in a multitude of ways. In various embodiments, a software module comprises a file, a section of code, a programming object, a programming structure, a distributed computing resource, a cloud computing resource, or combinations thereof. In further various embodiments, a software module comprises a plurality of files, a plurality of sections of code, a plurality of programming objects, a plurality of programming structures, a plurality of distributed computing resources, a plurality of cloud computing resources, or combinations thereof. In various embodiments, the one or more software modules comprise, by way of non-limiting examples, a web application, a mobile application, a standalone application, and a distributed or cloud computing application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on a distributed computing platform such as a cloud computing platform. In some embodiments, software modules are hosted on one or more machines in one location. In other embodiments, software modules are hosted on one or more machines in more than one location.

### Databases

In some embodiments, the platforms, systems, media, and methods disclosed herein include one or more databases, or use of the same. Various databases are suitable for storage and retrieval of information, for example customer incident data. In various embodiments, suitable databases include, by way of non-limiting examples, relational databases, non-relational databases, object oriented databases, object databases, entity-relationship model databases, associative databases, XML databases, document oriented databases, and graph databases. Further non-limiting examples include SQL, PostgreSQL, MySQL, Oracle, DB2, Sybase, and MongoDB. In some embodiments, a database is Internet-based. In further embodiments, a database is web-based. In still further embodiments, a database is cloud computing-based. In a particular embodiment, a database is a distributed database. In other embodiments, a database is based at least in part on one or more local computer storage devices.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-31. (canceled)

32. A method of treating a subject having or suspected of having hypertension or hyperlipidemia, comprising administering a nucleic acid molecule or a salt thereof configured to hybridize to a PDZK1 sequence to said subject.

33. The method of claim 32, wherein said nucleic acid molecule or salt thereof comprises a short interfering RNA (siRNA) molecule, a short hairpin RNA (shRNA) molecule, a microRNA adapted shRNA (shRNAmiR) molecule, a microRNA (miRNA molecule), or an antisense oligonucleotide (ASO), or any combination thereof.

34. The method of claim 33, wherein said nucleic acid molecule or salt thereof comprises said siRNA molecule, wherein said siRNA molecule comprises a duplex region of about 18 to about 27 base pairs.

35. The method of claim 32, wherein said PDZK1 sequence is a PDZK1 mRNA sequence.

36. The method of claim 32, wherein said nucleic acid molecule is not encapsulated in an exosome.

37. The method of claim 32, wherein said nucleic acid molecule comprises a sequence having at least 80% sequence identity to any one of SEQ ID NOs: 1-28.

38. The method of claim 32, wherein said subject has or is suspected of having hyperlipidemia, wherein said hyperlipidemia comprises hypercholesterolemia.

39. The method of claim 32, wherein said subject has or is suspected of having hyperlipidemia, wherein said hyperlipidemia comprises glyceridemia.

40. The method of claim 39, wherein said glyceridemia comprises hypertriglyceridemia.

41. The method of claim 32, wherein said subject has or is suspected of having hypertension.

42. A method of treating a subject having or suspected of having hyperlipidemia, comprising administering a nucleic acid molecule or a salt thereof to said subject, wherein said nucleic acid molecule comprises a sequence having at least 80% sequence identity to any one of SEQ ID NOs: 1-28, or a variant thereof.

43. The method of claim 42, wherein said hyperlipidemia comprises hypertriglyceridemia.

44. The method of claim 42, wherein said nucleic acid molecule comprises a sequence having at least 80% sequence identity to any one of SEQ ID NOs: 1, 2, 7, 10-12, or 14-28, or a variant thereof.

45. The method of claim 44, wherein said hyperlipidemia comprises hypercholesterolemia.

46. An siRNA molecule or a salt thereof having a duplex region 18-27 base pairs in length, wherein said duplex region comprises a sequence having at least 80% sequence identity to any one of SEQ ID NOs: 1-4, 7-9, 11, 15-28, or a variant thereof.

47. The siRNA molecule or the salt thereof of claim 46, wherein said duplex region comprises a sequence having at least 90% sequence identity to any one of SEQ ID NOs: 2, 7, 11, 15-28, or a variant thereof.

48. The siRNA molecule or the salt thereof of claim 46, wherein said duplex region is 20-27 base pairs in length.

49. The siRNA molecule or the salt thereof of claim 46, wherein said duplex region comprises a sequence having at least 80% sequence identity to any one of SEQ ID NOs: 1, 2, 3, 4, 8, 9, or 15-22, or 23-28, or a variant thereof.

50. The siRNA molecule or the salt thereof of claim 46, wherein said siRNA molecule comprises a non-natural RNA nucleotide or a non-natural linkage.

51. The siRNA molecule or the salt thereof of claim 46, wherein said siRNA molecule comprises a non-natural RNA nucleotide, wherein said non-natural RNA nucleotide comprises 2′-fluoro, 2′-O-methyl, 2¹-O-methyl, 2¹-O-methoxy-ethyl, 2¹-O-methoxy-ethoxy, 5′-methyl, SNA, hGNA, hhGNA, mGNA, TNA, h′GNA, a locked nucleic acid (LNA), or any combination thereof.


SEQUENCE LISTING (29 sequences)

SEQ ID NO: 1 - RNA, 21 nt, synthetic construct
  source 1..21: mol_type = other RNA
gcttcttcctgcgaattgaga

SEQ ID NO: 2 - RNA, 21 nt, synthetic construct
  source 1..21: mol_type = other RNA
gaagtcaggaagccgtgtcat

SEQ ID NO: 3 - RNA, 21 nt, synthetic construct
  source 1..21: mol_type = other RNA
gagcagaagatacaattcaaa

SEQ ID NO: 4 - RNA, 21 nt, synthetic construct
  source 1..21: mol_type = other RNA
ggtcaaatcatcaaggacata

SEQ ID NO: 5 - RNA, 21 nt, synthetic construct
  source 1..21: mol_type = other RNA
gaagaacaatgatctggtagt

SEQ ID NO: 6 - RNA, 21 nt, synthetic construct
  source 1..21: mol_type = other RNA
gcaggctggctaaaggtgaaa

SEQ ID NO: 7 - RNA, 21 nt, synthetic construct
  source 1..21: mol_type = other RNA
ggctcattcatcaaagaggta

SEQ ID NO: 8 - RNA, 21 nt, synthetic construct
  source 1..21: mol_type = other RNA
gtggaaagaaggcctatgatt

SEQ ID NO: 9 - RNA, 21 nt, synthetic construct
  source 1..21: mol_type = other RNA
ggaaagaaggcctatgattat

SEQ ID NO: 10 - RNA, 21 nt, synthetic construct
  source 1..21: mol_type = other RNA
ggctattgcagacggctaatt

SEQ ID NO: 11 - RNA, 21 nt, synthetic construct
  source 1..21: mol_type = other RNA
gctattgcagacggctaattt

SEQ ID NO: 12 - RNA, 21 nt, synthetic construct
  source 1..21: mol_type = other RNA
gcttaacttaggaagagataa

SEQ ID NO: 13 - RNA, 21 nt, synthetic construct
  source 1..21: mol_type = other RNA
ggaagagataaggcaagagct

SEQ ID NO: 14 - RNA, 21 nt, synthetic construct
  source 1..21: mol_type = other RNA
gagataaggcaagagctagat

SEQ ID NO: 15 - RNA, 21 nt, synthetic construct
  source 1..21: mol_type = other RNA
gtaggtcgccatcttgctata

SEQ ID NO: 16 - RNA, 21 nt, synthetic construct
  source 1..21: mol_type = other RNA
gatggtcagatccgcagagta

SEQ ID NO: 17 - RNA, 21 nt, synthetic construct
  source 1..21: mol_type = other RNA
ggaacaacagtatagaacata

SEQ ID NO: 18 - RNA, 21 nt, synthetic construct
  source 1..21: mol_type = other RNA
gacctaatcatagcgatagaa

SEQ ID NO: 19 - RNA, 21 nt, synthetic construct
  source 1..21: mol_type = other RNA
ggattaagcgatgacgtataa

SEQ ID NO: 20 - RNA, 21 nt, synthetic construct
  source 1..21: mol_type = other RNA
ggtgcgggacgacaatgaata

SEQ ID NO: 21 - RNA, 21 nt, synthetic construct
  source 1..21: mol_type = other RNA
ggatatggtcgccaacttaaa

SEQ ID NO: 22 - RNA, 21 nt, synthetic construct
  source 1..21: mol_type = other RNA
gtgaagtagcgaagtcatgta

SEQ ID NO: 23 - RNA, 21 nt, synthetic construct
  source 1..21: mol_type = other RNA
ggaatgattaaggtcgataca

SEQ ID NO: 24 - RNA, 21 nt, synthetic construct
  source 1..21: mol_type = other RNA
gtaagtcgtcacggtagtact

SEQ ID NO: 25 - RNA, 21 nt, synthetic construct
  source 1..21: mol_type = other RNA
gacttggcacttagcgtatat

SEQ ID NO: 26 - RNA, 21 nt, synthetic construct
  source 1..21: mol_type = other RNA
gaacaagaacgattagttagt

SEQ ID NO: 27 - RNA, 21 nt, synthetic construct
  source 1..21: mol_type = other RNA
gaagagcagacgtaggagtaa

SEQ ID NO: 28 - RNA, 21 nt, synthetic construct
  source 1..21: mol_type = other RNA
gacgacaaagtagggaatgat

SEQ ID NO: 29 - RNA, 2040 nt, Homo sapiens
  source 1..2040: mol_type = mRNA
gaattccgggcagctcctcttccatctccagaaatgacctccaccttcaacccccgagaatgtaaactgtccaagcaagaagggcaaaactatggcttcttcctgcgaattgagaaggacaccgagggccacctggtccgggtggttgagaagtgtagcccagcagagaaggctggccttcaagatggagacagagttcttaggatcaatggtgtctttgtggacaaagaagaacatatgcaggttgtggatctggtcagaaagagtgggaattcagtgactttactagttctggatggggattcctatgagaaagcagtgaaaacacgggtggacttgaaagagttgggtcaaagtcagaaggagcaaggtttgagtgataatatactttcccctgtgatgaatggaggtgtgcaaacttggacccagccccggctctgctatctcgtgaaggaaggaggcagctatggcttctctctgaaaactgtccaaggtaaaaagggggtgtacatgactgatattacacctcaaggtgtggctatgagagctggagttctggctgatgatcacttgattgaagtgaatggagagaatgtagaggatgccagccatgagaaagtggttgaaaaggtgaagaagtcaggaagccgtgtcatgttcctgctggtggacaaagaaactgacaagcgtcatgttgagcagaagatacaattcaaaagagaaacagccagtttgaaactgttaccccaccagccccgaattgtggagatgaagaaaggaagcaatggctatggtttctatctgagggcaggctcagaacagaaaggtcaaatcatcaaggacatagattctggaagtccagcagaggaggctggcttgaagaacaatgatctggtagttgctgtcaacggcgagtctgtggaaaccctggatcatgacagtgtggtagaaatgattagaaagggtggagatcagacttcactgttggtggtagacaaagagacggacaacatgtacagactggctcatttttctccatttctctactatcaaagtcaagaactgcccaatggctctgtcaaggaggctccagctcctactcccacttctctggaagtctcaagtccaccagatactacagaggaagtagatcataagcctaaactctgcaggctggctaaaggtgaaaatggctatggctttcacttaaatgcgattcggggtctgccaggctcattcatcaaagaggtacagaagggcggtcctgctgacttggctgggctagaggatgaggatgtcatcattgaagtgaatggggtgaatgtgctagatgaaccctatgagaaggtggtggatagaatccagagcagtgggaagaatgtcacacttctagtctgtggaaagaaggcctatgattatttccaagctaagaaaatccctattgtttcctccctggctgatccacttgacacccctccagattctaaagaaggaatagtggtggagtcaaaccatgactcgcacatggcaaaagaacgggcccacagtacagcctcacattcttcttccaattctgaagatacagagatgtgatgaaaacaagtaatagctttggctgtttatttgatagctgtttctgggtatttaataggaatcctttctcaaggaatgagttgtgacctgtttactgtctctttagaagaaaaactccactggaaaccattcaccatgtgtgactgtcttctgttatcatttgtcttacaggcggctattgcagacggctaatttatgcttaacttaggaagagataaggcaagagctagatttttttcatgtgatcttttccaagcttcaacttaacttaactacatttctctgtatgatgatgtctcttacttctacaggttccttgagcaccaaagatgattcataactctgtataggtgacagctgcttataaaagcatcttagcagataagcctattaaaattgtgcttttgtaacaatgttgtggttgctagaataaataccatgaacccg